(19) United States
(12) Patent Application Publication
(10) Pub. No.: US 2020/0333313 A1
(43) Pub. Date: Oct. 22, 2020

(54) Method for Calculating Saturation of Natural Gas Hydrate Based on Wood Wave Impedance Method

(71) Applicants: Guangdong University of Petrochemical Technology, Maoming City (CN); Chengdu University of Technology, Chengdu (CN)

(72) Inventors: Xiangsheng Bao, Maoming City (CN); Haiyan Zhou, Maoming City (CN); Jianrong Ding, Maoming City (CN)

(21) Appl. No.: 16/562,664

(22) Filed: Sep. 6, 2019

(30) Foreign Application Priority Data
Apr. 22, 2019 (CN) 201910321244.2

(51) Int. Cl. G01N 33/22; G01N 3/08; G01N 27/02; G06F 17/13

(52) U.S. Cl. CPC G01N 33/225; G06F 17/13; G01N 27/02; G01N 3/08

(57) ABSTRACT

In a method for calculating saturation of a natural gas hydrate based on a Wood wave impedance method a compressional wave impedance Z_{b} of a deposit containing the natural gas hydrate can be obtained by compressional wave impedance inversion, and a compressional wave impedance Z_{w} of the fluid and a compressional wave impedance Zₕ of the pure natural gas hydrate can be calculated by measuring relevant elastic parameters in a laboratory, a compressional wave impedance Zₘ of a matrix can be calculated on the basis of drilling data and measurement data of the relevant elastic parameters measured in the laboratory, and a porosity Φ can be obtained by utilizing a logging interpretation technique, and the saturation of the natural gas hydrate can be calculated.

### CROSS-REFERENCE TO RELATED APPLICATION(S)

This application claims priority to Chinese application number 201910321244.2, filed Apr. 22, 2019, with a title of METHOD FOR CALCULATING SATURATION OF NATURAL GAS HYDRATE BASED ON WOOD WAVE IMPEDANCE METHOD. The above-mentioned patent application is incorporated herein by reference in its entirety.

### TECHNICAL FIELD

The present invention relates to the technical field of research on degrees of enrichment of natural gas hydrates, and in particular to a method for calculating saturation of a natural gas hydrate based on a Wood wave impedance method.

### BACKGROUND

A natural gas hydrate is a cage compound formed by water and natural gas under low temperature and high pressure, and is an ice-like substance. The natural gas hydrate has no fixed chemical formula and is a non-stoichiometric mixture. The natural gas hydrate is mainly distributed in land permafrost and seabed deposits. Products obtained by combustion of the natural gas hydrate are water and carbon dioxide. The natural gas hydrate is an efficient and clean unconventional energy source and can be used as an important replacement energy source for fossil energy in the future. Natural gas hydrates are mostly distributed in seas, and the natural gas hydrates in the seas account for 98% of the total natural gas hydrates worldwide.

Studies have shown that natural gas hydrates are distributed in a stabilized zone formed by temperature and pressure, but the stabilized zone only determines the spatial range of the natural gas hydrates. The enrichment of the natural gas hydrates in a certain region in the stabilized zone is also affected by constraints such as gas source conditions, gas migration conditions and reservoir conditions. At present, seismic methods used to predict a degree of enrichment of a natural gas hydrate in a certain region are mainly: a BSR method, an amplitude blanking zone method, an attribute prediction method, a wave impedance prediction method, a hydrate saturation prediction method, and the like. These methods are divided into three categories according to quantitative features: (1) qualitative detection methods such as the BSR method and the amplitude blanking zone method: these methods have the advantages of being visual, easy to use and easy to understand, but the degree of quantification is not enough, which is not conducive to judging the difference in degrees of enrichment of natural gas hydrates at different locations; (2) semi-quantitative prediction methods, such as the attribute prediction method: these methods have certain quantitative features, and some quantitative methods can initially reflect the difference in degrees of enrichment of natural gas hydrates, but the reflected difference in degrees of enrichment is also relative; (3) quantitative prediction methods, such as the wave impedance prediction method and the hydrate saturation prediction method: these methods are the best methods to better reflect the difference in degrees of enrichment of natural gas hydrates. Therefore, among the three types of methods, the quantitative prediction methods are the most popular methods for predicting the degree of enrichment of hydrates in practical applications. Among the quantitative prediction methods, the hydrate saturation prediction method is most relevant to the calculation of hydrate resource quantity, so this method is of great significance for the commercialization process of natural gas hydrates in a region.

The three most classical methods for hydrate saturation prediction are a Timur method, a Wood method, and a Gassmann method. Each of the three methods has its own applicable conditions. In the sea environment, natural gas hydrates exist in the marine deposits in three main modes: a suspension mode, a particle contact mode and a cementation mode. Natural gas hydrates in the Shenhu sea area of China are mainly in the suspension mode. The Wood method is currently a better method for predicting the saturation of natural gas hydrates in the suspension mode. From the formation of zero-offset seismic data, the seismic data can be seen as being formed by convolution of a reflection sequence formed by the impedance difference underground and seismic wavelets. The post-stack seismic inversion can directly reverse the wave impedance, natural gas hydrates of different saturations will cause the wave impedance to change, but the Wood method does not give the relationship between natural gas hydrates of different saturations and wave impedance. Therefore, it is difficult to apply the Wood method directly to the actual seismic data to predict the saturation of the natural gas hydrates.

In summary, there is an urgent need to improve the Wood method to form a method for predicting saturation of a natural gas hydrate in the suspension mode.

### SUMMARY

The present invention provides a method for calculating saturation of a natural gas hydrate based on a Wood wave impedance method, which solves the problem that it is difficult to predict the natural gas hydrate saturation by using a Wood method in the prior art in practice.

The technical solution of the present invention is implemented as follows:

A method for calculating saturation of a natural gas hydrate based on a Wood wave impedance method is provided, and the method includes the following steps:

(1) a Wood method for obtaining saturation of the natural gas hydrate consisting of an equation

$$\frac{1}{{ρ}_{b} {V}_{b}^{2}} = \frac{Φ ( 1 - {S}_{h} )}{{ρ}_{w} {V}_{p w}^{2}} + \frac{Φ {S}_{h}}{{ρ}_{h} {V}_{p h}^{2}} + \frac{1 - Φ}{{ρ}_{m} {V}_{p m}^{2}}$$

and an equation ρ_{b}=(1−Sₕ)Φρ_{w}+ΦSₕρₕ+(1−Φ)ρₘ, where V_{b}, V_{pw}, Vₚₕ, and Vₚₘ represent the compressional wave velocity of a deposit containing the natural gas hydrate, the compressional wave velocity of a fluid, the compressional wave velocity of a pure natural gas hydrate and the compressional wave velocity of a matrix, respectively; Φ represents porosity; Sₕ represents the proportion of the natural gas hydrate in a pore space, and ρ_{b}, ρ_{w}, ρₕ and ρₘ represent the density of the deposit containing the natural gas hydrate, the density of the fluid, the density of the pure natural gas hydrate, and the density of the matrix, respectively;

(2) a formula for calculating the matrix density being

$${ρ}_{m} = {\sum }_{i = 1}^{n} {f}_{i} {ρ}_{i} ,$$

and a formula for calculating the compressional wave velocity of the matrix being

$${V}_{p m} = \sqrt{\frac{K + \frac{4}{3} G}{{ρ}_{m}}} ;$$

where fᵢ is the volume percentage of an i-th substance in the matrix, ρᵢ is the density of the i-th substance in the matrix, n represents the kind of a substance forming the matrix, K represents a substance bulk modulus, G represents a substance shear modulus,

$$K = \frac{1}{2} [ {\sum }_{i = 1}^{n} {f}_{i} {K}_{i} + {( {\sum }_{i = 1}^{n} \frac{{f}_{i}}{{K}_{i}} )}^{- 1} ] , \\ G = \frac{1}{2} [ {\sum }_{i = 1}^{n} {f}_{i} {G}_{i} + {( {\sum }_{i = 1}^{n} \frac{{f}_{i}}{{G}_{i}} )}^{- 1} ] ,$$

where Kᵢ is the bulk modulus of the i-th substance in the matrix, and Gᵢ is the shear modulus of the i-th substance in the matrix;

(3) a formula for calculating the compressional wave velocity of a pure natural gas hydrate being

$${V}_{p h} = \sqrt{\frac{E ( 1 - σ )}{ρ ( 1 + σ ) ( 1 - 2 σ )}} ,$$

where E is the Young's modulus of the pure natural gas hydrate, ρ is the density of the pure natural gas hydrate, and σ is the Poisson's ratio of the pure natural gas hydrate; where the Young's modulus is obtained by a formula

$$E = \frac{9 K G}{3 K + G} ,$$

and the Poisson's ratio is obtained by a formula

$$σ = \frac{3 K - 2 G}{2 ( 3 K + G )} ;$$

(4) multiplying both sides of the equation

$$\frac{1}{{ρ}_{b} {V}_{b}^{2}} = \frac{Φ ( 1 - {S}_{h} )}{{ρ}_{w} {V}_{p w}^{2}} + \frac{Φ {S}_{h}}{{ρ}_{h} {V}_{p h}^{2}} + \frac{1 - Φ}{{ρ}_{m} {V}_{p m}^{2}}$$

by

$$\frac{1}{{ρ}_{b}}$$

simultaneously to obtain an equation

$$\frac{1}{{( {ρ}_{b} {V}_{b} )}^{2}} = \frac{Φ ( 1 - {S}_{h} )}{{ρ}_{b} {ρ}_{w} {V}_{p w}^{2}} + \frac{Φ {S}_{h}}{{ρ}_{b} {ρ}_{h} {V}_{p h}^{2}} + \frac{1 - Φ}{{ρ}_{b} {ρ}_{m} {V}_{p m}^{2}} ;$$

where the compressional wave impedance of the deposit containing the natural gas hydrate is Z_{b}=ρ_{b}V_{b}, the compressional wave impedance of the fluid is Z_{w}=ρ_{w}V_{pw}, the compressional wave impedance of the pure natural gas hydrate is Zₕ=ρₕVₚₕ, the compressional wave impedance of the matrix is Zₘ=ρₘVₚₘ, and then the equation

$$\frac{1}{{( {ρ}_{b} {V}_{b} )}^{2}} = \frac{Φ ( 1 - {S}_{h} )}{{ρ}_{b} {ρ}_{w} {V}_{p w}^{2}} + \frac{Φ {S}_{h}}{{ρ}_{b} {ρ}_{h} {V}_{p h}^{2}} + \frac{1 - Φ}{{ρ}_{b} {ρ}_{m} {V}_{p m}^{2}}$$

can be expressed as an equation

$$\frac{1}{{( {Z}_{b} )}^{2}} = \frac{Φ ( 1 - {S}_{h} )}{( {ρ}_{b} \text{/} {ρ}_{w} ) {( {Z}_{w} )}^{2}} + \frac{Φ {S}_{h}}{( {ρ}_{b} \text{/} {ρ}_{h} ) {( {Z}_{h} )}^{2}} + \frac{1 - Φ}{( {ρ}_{b} \text{/} {ρ}_{m} ) {( {Z}_{m} )}^{2}} ;$$

multiplying both sides of an equation ρ_{b}=(1−Sₕ)Φρ_{w}+ΦSₕρₕ+(1−Φ)ρₘ by

$$\frac{1}{{ρ}_{w}}$$

simultaneously to obtain an equation ρ_{b}/ρ_{w}=(1−Sₕ)Φ+ΦSₕρₕ/ρ_{w}+(1−Φ)ρₘ/ρ_{w}, multiplying both sides of an equation ρ_{b}=(1−Sₕ)Φρ_{w}+ΦSₕρₕ+(1−Φ)ρₘ by

$$\frac{1}{{ρ}_{h}}$$

simultaneously to obtain an equation ρ_{b}/ρₕ=(1−Sₕ)Φ+ρ_{w}/ρₕ+Sₕ+(1−Φ)ρₘ/ρₕ, and multiplying both sides of an equation ρ_{b}=(1−Sₕ)Φρ_{w}+ΦSₕρₕ+(1−Φ)ρₘ by

$$\frac{1}{{ρ}_{m}}$$

simultaneously to obtain an equation ρ_{b}/ρₘ=(1−Sₕ)Φρ_{w}/ρₘ+ΦSₕρₕ/ρₘ+(1−Φ), and setting C_{bw}=ρ_{b}/ρ_{w}, C_{bh}=ρ_{b}/ρₕ, C_{bm}=ρ_{b}/ρₘ, and C_{bh} [symbol] C_{bw} [symbol] 1 [symbol] C_{bm};

(5) substituting C_{bw}, C_{bh} and C_{bm} into an equation

$$\frac{1}{{( {Z}_{b} )}^{2}} = \frac{Φ ( 1 - {S}_{h} )}{( {ρ}_{b} / {ρ}_{w} ) {( {Z}_{w} )}^{2}} + \frac{Φ {S}_{h}}{( {ρ}_{b} / {ρ}_{h} ) {( {Z}_{h} )}^{2}} + \frac{1 - Φ}{( {ρ}_{b} / {ρ}_{m} ) {( {Z}_{m} )}^{2}} ,$$

to obtain a formula

$$\frac{1}{{( {Z}_{b} )}^{2}} = \frac{Φ ( 1 - {S}_{h} )}{{{C}_{b w} ( {Z}_{w} )}^{2}} + \frac{Φ {S}_{h}}{{{C}_{b h} ( {Z}_{h} )}^{2}} + \frac{1 - Φ}{{{C}_{b m} ( {Z}_{m} )}^{2}}$$

for calculating the saturation of the natural gas hydrate by using a Wood wave impedance method, where the compressional wave impedance Z_{b} of the deposit containing the natural gas hydrate can be obtained by compressional wave impedance inversion, and the compressional wave impedance Z_{w} of the fluid and the compressional wave impedance Zₕ of the pure natural gas hydrate can be calculated by measuring relevant elastic parameters in a laboratory; the compressional wave impedance Zₘ of the matrix can be calculated on the basis of drilling data and measurement data of the relevant elastic parameters measured in the laboratory, and the porosity Φ can be obtained by utilizing a logging interpretation technique.

The beneficial effects of the present invention are:

The post-stack inversion workload is small, and the requirements for interpreting staff are not high. The relationship between wave impedance and the saturation of the natural gas hydrate can be well established, which is of great significance for the estimation of natural gas hydrate reservoirs in the sea area in China.

The method of the present invention forms a novel prediction method by deriving and analyzing the existing Wood method, and clearly shows the relationship between the compressional wave impedance of the natural gas hydrate reservoir and the saturation of the natural gas hydrate, and the method has a small error and has a certain promotion and application value.

The present summary is provided only by way of example, and not limitation. Other aspects of the present invention will be appreciated in view of the entirety of the present disclosure, including the entire text, claims and accompanying figure(s).

### BRIEF DESCRIPTION OF THE DRAWINGS

To describe the technical solutions in the embodiments of the present invention or in the prior art more clearly, the following briefly describes the accompanying drawings required for describing the embodiments or the prior art. Apparently, the accompanying drawings in the following description show some embodiments of the present invention, and a person of ordinary skill in the art may still derive other drawings from these accompanying drawings without creative efforts.

FIG. 1 shows elastic parameters of a deposit matrix composition of a natural gas hydrate-enriched zone in the Shenhu sea area.

### DETAILED DESCRIPTION OF EMBODIMENTS

The following describes technical solutions of one or more embodiments of the present invention with reference to the accompanying drawing(s). Apparently, the described embodiment(s) are merely a part rather than all of the embodiments of the present invention. All other embodiments obtained by a person of ordinary skill in the art based on the embodiments of the present invention without creative efforts shall fall within the protection scope of the present invention. All other embodiments obtained by a person of ordinary skill in the art based on the embodiments of the present invention without creative efforts shall fall within the protection scope of the present invention.

Taking the calculation of saturation of a natural gas hydrate in the Shenhu sea area of China as an example, a method for calculating saturation of a natural gas hydrate based on a Wood wave impedance method includes the following steps:

Step (1): a Wood method for obtaining saturation of the natural gas hydrate consists of an equation

$$\frac{1}{{ρ}_{b} {V}_{b}^{2}} = \frac{Φ ( 1 - {S}_{h} )}{{ρ}_{w} {V}_{p w}^{2}} + \frac{Φ {S}_{h}}{{ρ}_{h} {V}_{p h}^{2}} + \frac{1 - Φ}{{ρ}_{m} {V}_{p m}^{2}}$$

and an equation ρ_{b}=(1−Sₕ)Φρ_{w}+ΦSₕρₕ+(1−Φ)ρₘ, where V_{b}, V_{pw}, Vₚₕ, and Vₚₘ represent the compressional wave velocity of a deposit containing the natural gas hydrate, the compressional wave velocity of a fluid, the compressional wave velocity of a pure natural gas hydrate and the compressional wave velocity of a matrix, respectively; Φ represents porosity; Sₕ represents the proportion of the natural gas hydrate in a pore space, and ρ_{b}, ρ_{w}, ρₕ and ρₘ represent the density of the deposit containing the natural gas hydrate, the density of the fluid, the density of the pure natural gas hydrate, and the density of the matrix, respectively.

Step (2): since the matrix is often composed of many substances, a formula for calculating the matrix density can be expressed as

$${ρ}_{m} = {\sum }_{i = 1}^{n} {f}_{i} {ρ}_{i} ,$$

and a formula for calculating the compressional wave velocity of the matrix is

$${V}_{p m} = \sqrt{\frac{K + \frac{4}{3} G}{{ρ}_{m}}} ;$$

where fᵢ is the volume percentage of an i-th substance in the matrix, ρᵢ is the density of the i-th substance in the matrix, n represents the kind of a substance forming the matrix, K represents a substance bulk modulus, G represents a substance shear modulus,

$$K = \frac{1}{2} [ {\sum }_{i = 1}^{n} {f}_{i} {K}_{i} + {( {\sum }_{i = 1}^{n} \frac{{f}_{i}}{{K}_{i}} )}^{- 1} ] , G = \frac{1}{2} [ {\sum }_{i = 1}^{n} {f}_{i} {G}_{i} + {( {\sum }_{i = 1}^{n} \frac{{f}_{i}}{{G}_{i}} )}^{- 1} ] ,$$

where Kᵢ is the bulk modulus of the i-th substance in the matrix, and Gᵢ is the shear modulus of the i-th substance in the matrix.

Step (3): a formula for calculating the compressional wave velocity of a pure natural gas hydrate is

$${V}_{p h} = \sqrt{\frac{E ( 1 - σ )}{ρ ( 1 + σ ) ( 1 - 2 σ )}} ,$$

where E is the Young's modulus of the pure natural gas hydrate, ρ is the density of the pure natural gas hydrate, and σ is the Poisson's ratio of the pure natural gas hydrate; where the Young's modulus is obtained by a formula

$$E = \frac{9 K G}{3 K + G} ,$$

and the Poisson's ratio is obtained by a formula

$$σ = \frac{3 K - 2 G}{2 ( 3 K + G )} .$$

Step (4): multiply both sides of the equation

$$\frac{1}{{ρ}_{b} {V}_{b}^{2}} = \frac{Φ ( 1 - {S}_{h} )}{{ρ}_{w} {V}_{p w}^{2}} + \frac{Φ {S}_{h}}{{ρ}_{h} {V}_{p h}^{2}} + \frac{1 - Φ}{{ρ}_{m} {V}_{p m}^{2}}$$

by

$$\frac{1}{{ρ}_{b}}$$

to obtain an equation

$$\frac{1}{{( {ρ}_{b} {V}_{b} )}^{2}} = \frac{Φ ( 1 - {S}_{h} )}{{ρ}_{b} {ρ}_{w} {V}_{p w}^{2}} + \frac{Φ {S}_{h}}{{ρ}_{b} {ρ}_{h} {V}_{p h}^{2}} + \frac{1 - Φ}{{ρ}_{b} {ρ}_{m} {V}_{p m}^{2}} ;$$

where the compressional wave impedance of the deposit containing the natural gas hydrate is Z_{b}=ρ_{b}V_{b}, the compressional wave impedance of the fluid is Z_{w}=ρ_{w}V_{pw}, the compressional wave impedance of the pure natural gas hydrate is Zₕ=ρₕVₚₕ, the compressional wave impedance of the matrix is Zₘ=ρₘVₚₘ, and then the equation

$$\frac{1}{{( {ρ}_{b} {V}_{b} )}^{2}} = \frac{Φ ( 1 - {S}_{h} )}{{ρ}_{b} {ρ}_{w} {V}_{p w}^{2}} + \frac{Φ {S}_{h}}{{ρ}_{b} {ρ}_{h} {V}_{p h}^{2}} + \frac{1 - Φ}{{ρ}_{b} {ρ}_{m} {V}_{p m}^{2}}$$

can be expressed as an equation

$$\frac{1}{{( {Z}_{b} )}^{2}} = \frac{Φ ( 1 - {S}_{h} )}{( {ρ}_{b} / {ρ}_{w} ) {( {Z}_{w} )}^{2}} + \frac{Φ {S}_{h}}{( {ρ}_{b} / {ρ}_{h} ) {( {Z}_{h} )}^{2}} + \frac{1 - Φ}{( {ρ}_{b} / {ρ}_{m} ) {( {Z}_{m} )}^{2}} .$$

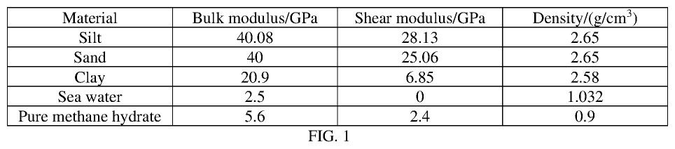

The deposit matrix of a natural gas hydrate-enriched zone in the Shenhu sea area is mainly composed of silt, sand and clay, and also includes seawater and pure methane hydrate. FIG. 1 shows elastic parameters of a deposit matrix composition actually measured. Two sides of the equation ρ_{b}=(1−Sₕ)Φρ_{w}+ΦSₕρₕ+(1−Φ)ρₘ are multiplied by

$$\frac{1}{{ρ}_{w}}$$

simultaneously to obtain an equation ρ_{b}/ρ_{w}=(1−Sₕ)Φ+ΦSₕρₕ/ρ_{w}+(1−Φ)ρₘ/ρ_{w}, and ρ_{b}/ρ_{w}≈(1−Sₕ)Φ+0.87ΦSₕ+0.97(1−Φ)ρₘ=0.97(1−Φ)ρₘ+Φ−0.13ΦSₕ can be obtained by substituting elastic parameters in FIG. 1.

Both sides of the equation ρ_{b}=(1−Sₕ)Φρ_{w}+ΦSₕρₕ+(1−Φ)ρₘ are multiplied by

$$\frac{1}{{ρ}_{h}}$$

simultaneously to obtain an equation ρ_{b}/ρₕ=(1−Sₕ)Φρ_{w}/ρₕ+ΦSₕ+(1−Φ)ρₘ/ρₕ, and ρ_{b}/ρₕ≈1.15(1−Sₕ)Φ+ΦSₕ+1.11(1−Φ)ρₘ=1.11(1−Φ)ρₘ+1.15Φ+0.15ΦSₕ can be obtained by substituting the elastic parameters in FIG. 1.

Both sides of the equation ρ_{b}=(1−Sₕ)Φρ_{w}+ΦSₕρₕ+(1−Φ)ρₘ are multiplied by

$$\frac{1}{{ρ}_{m}}$$

simultaneously to obtain an equation ρ_{b}/ρₕ=(1−Sₕ)Φρ_{w}/ρₘ+ΦSₕρₕ/ρₘ+(1−Φ), and ρ_{b}/ρₘ≈(1−Φ)+1.03Φ/ρₘ−0.13ΦSₕ/ρₘ can be obtained by substituting the elastic parameters in FIG. 1.

Set C_{bw}=ρ_{b}/ρ_{w}, C_{bh}=ρ_{b}/ρₕ, C_{bm}=ρ_{b}/ρₘ, and C_{bh} [symbol] C_{bw} [symbol] 1 [symbol] C_{bm}, since ρ_{b} is generally greater than 1.5 g/cm³ and the maximum matrix density generally does not exceed 3 g/cm³, the smallest coefficient C_{bm} is greater than 0.5, and ΦSₕ generally is about 0.1. Relative to the value greater than 0.5, the value of ΦSₕ is negligible, and then C_{bw}≈0.97(1−Φ)ρₘ+Φ, C_{bh}≈1.11(1−Φ)ρₘ+1.15Φ, C_{bm}≈(1−Φ)+1.03Φ/ρₘ, C_{bw}, C_{bh}, and C_{bm} can be considered as a coefficient related to porosity and matrix density.

Step (5): substitute C_{bw}, C_{bh} and C_{bm} into an equation

$$\frac{1}{{( {Z}_{b} )}^{2}} = \frac{Φ ( 1 - {S}_{h} )}{( {ρ}_{b} / {ρ}_{w} ) {( {Z}_{w} )}^{2}} + \frac{Φ {S}_{h}}{( {ρ}_{b} / {ρ}_{h} ) {( {Z}_{h} )}^{2}} + \frac{1 - Φ}{( {ρ}_{b} / {ρ}_{m} ) {( {Z}_{m} )}^{2}} ,$$

to obtain a formula

$$\frac{1}{{( {Z}_{b} )}^{2}} = \frac{Φ ( 1 - {S}_{h} )}{{{C}_{b w} ( {Z}_{w} )}^{2}} + \frac{Φ {S}_{h}}{{{C}_{b h} ( {Z}_{h} )}^{2}} + \frac{1 - Φ}{{{C}_{b m} ( {Z}_{m} )}^{2}}$$

for calculating the saturation of the natural gas hydrate by using a Wood wave impedance method, where the compressional wave impedance Z_{b} of the deposit containing the natural gas hydrate can be obtained by compressional wave impedance inversion, and the compressional wave impedance Z_{w} of the fluid and the compressional wave impedance Zₕ of the pure natural gas hydrate can be calculated by measuring relevant elastic parameters in a laboratory; the compressional wave impedance Zₘ of the matrix can be calculated on the basis of drilling data and measurement data of the relevant elastic parameters measured in the laboratory, and the porosity Φ can be obtained by utilizing a logging interpretation technique.

In order to verify the reliability of the method of the present invention, an error analysis is performed on the above method:

First, some basic data assumptions are made. It is assumed that the matrix of natural gas hydrate deposit in the sea area is composed of siltstone and clay, and their proportions in the matrix is 75% and 25%, respectively; the natural gas hydrate in a suspension mode is generally less than 50%, it is assumed that the saturation of the natural gas hydrate for the study is 30%; and it is assumed that the porosity of the natural gas hydrate deposit is 40%.

From FIG. 1 and the formula

$${ρ}_{m} = {\sum }_{i = 1}^{n} {f}_{i} {ρ}_{i} ,$$

the density of the matrix can be calculated to be about 2.63 g/cm³. From FIG. 1 and the formulas

$$K = \frac{1}{2} [ {\sum }_{i = 1}^{n} {f}_{i} {K}_{i} + {( {\sum }_{i = 1}^{n} \frac{{f}_{i}}{{K}_{i}} )}^{- 1} ] and$$

$$G = \frac{1}{2} [ {\sum }_{i = 1}^{n} {f}_{i} {G}_{i} + {( {\sum }_{i = 1}^{n} \frac{{f}_{i}}{{G}_{i}} )}^{- 1} ] ,$$

the bulk modulus and shear modulus of the matrix can be calculated to be about 33.94 GPa and 19.32 GPa, respectively; and from the matrix density and the bulk modulus and the shear modulus, the compressional wave velocity of the matrix can be calculated to be 4762.34 m/s using a formula

$${V}_{p m} = \sqrt{\frac{K + \frac{4}{3} G}{{ρ}_{m}}} .$$

From FIG. 1 and the formulas

$$E = \frac{9 K G}{3 K + G} and$$

$$σ = \frac{3 K - 2 G}{2 ( 3 K + G )} ,$$

the Young's modulus and Poisson's ratio of the natural gas hydrate can be calculated to be about 6.3 GPa and 0.31, respectively. The compressional wave velocity of the natural gas hydrate can be calculated from FIG. 1 and the formula

$${V}_{p h} = \sqrt{\frac{E ( 1 - σ )}{ρ ( 1 + σ ) ( 1 - 2 σ )}}$$

to be 3126.94 m/s; the coefficients C_{bw}, C_{bh} and C_{bm} are further calculate to be 1.93, 2.21 and 0.76 respectively; the density of the deposit containing the natural gas hydrate can be calculated to be about 1.97 g/cm³ according to the formula ρ_{b}=(1−Sₕ)Φρ_{w}+ΦSₕρₕ+(1−Φ)ρₘ, and it can be calculated according to the formula

$$\frac{1}{{ρ}_{b} {V}_{b}^{2}} = \frac{Φ ( 1 - {S}_{h} )}{{ρ}_{w} {V}_{p w}^{2}} + \frac{Φ {S}_{h}}{{ρ}_{h} {V}_{p h}^{2}} + \frac{1 - Φ}{{ρ}_{m} {V}_{p m}^{2}}$$

that the compressional wave velocity of the deposit containing the natural gas hydrate is about 1855.96 m/s. It can be known in combination with the calculated data that the compressional wave impedance Z_{b} of the deposit containing the natural gas hydrate, the compressional wave impedance Z_{w} of the fluid, the compressional wave impedance Zₕ of the pure natural gas hydrate, and the compressional wave impedance Zₘ of the matrix are about 3651.61 (m·g)/(s·cm³), 12536.88 (m·g)/(s·cm³), 1527.36 (m·g)/(s·cm³), and 2814.25 (m·g)/(s·cm³) respectively; and it is further calculated according to the formula

$$\frac{1}{{( {Z}_{b} )}^{2}} = \frac{Φ ( 1 - {S}_{h} )}{{{C}_{b w} ( {Z}_{w} )}^{2}} + \frac{Φ {S}_{h}}{{{C}_{b h} ( {Z}_{h} )}^{2}} + \frac{1 - Φ}{{{C}_{b m} ( {Z}_{m} )}^{2}}$$

that the saturation of the natural gas hydrate is 28.5%.

The accurate saturation of the natural gas hydrate obtained by the actual measurement is 30%, and it can be seen that the saturation value of the natural gas hydrate calculated by the method of the present invention is very close to the actual value, and the error is small.

In conclusion, the method of the present invention forms a novel prediction method by deriving and analyzing the existing Wood method, and clearly shows the relationship between the compressional wave impedance of the natural gas hydrate reservoir and the saturation of the natural gas hydrate, and the method has a small error and has a certain promotion and application value.

The above-mentioned contents are merely preferred embodiments of the present invention, and are not used to limit the present invention, and wherever within the spirit and principle of the present invention, any modifications, equivalent replacements, improvements, and the like shall be all contained within the protection scope of the present invention.

1. A method for calculating saturation of a natural gas hydrate based on a Wood wave impedance method, the method comprising:
(1) using a Wood method to predict a saturation of the natural gas hydrate utilizing an equation and an equation
$$\frac{1}{{ρ}_{b} {V}_{b}^{2}} = \frac{Φ ( 1 - {S}_{h} )}{{ρ}_{w} {V}_{p w}^{2}} + \frac{Φ {S}_{h}}{{ρ}_{h} {V}_{p h}^{2}} + \frac{1 - Φ}{{ρ}_{m} {V}_{p m}^{2}}$$
and an equation ρ_{b}=(1−Sₕ)Φρ_{w}+ΦSₕρₕ+(1−Φ)ρₘ, wherein V_{b}, V_{pw}, Vₚₕ, and Vₚₘ represent a compressional wave velocity of a deposit containing the natural gas hydrate, compressional wave velocity of a fluid, a compressional wave velocity of a pure natural gas hydrate and a compressional wave velocity of a matrix of the deposit, respectively; Φ represents porosity; Sₕ represents a proportion of the natural gas hydrate in a pore space, and ρ_{b}, ρ_{w}, ρₕ and ρₘ represent a bulk density of the deposit containing the natural gas hydrate, a density of the fluid, a density of the pure natural gas hydrate, and a density of the matrix of the deposit, respectively;
(2) calculating the density of the matrix of the deposit utilizing a formula
$${ρ}_{m} = {\sum }_{i = 1}^{n} {f}_{i} {ρ}_{i} ,$$
and calculating the compressional wave velocity of the matrix of the deposit utilizing a formula
$${V}_{p m} = \sqrt{\frac{K + \frac{4}{3} G}{{ρ}_{m}}} ;$$
wherein fᵢ is a volume percentage of an i-th substance in the matrix of the deposit, ρᵢ is a density of the i-th substance in the matrix of the deposit, n represents the kinds of a substances forming the matrix of the deposit, K represents a substance bulk modulus, G represents a substance shear modulus,
$$K = \frac{1}{2} [ {\sum }_{i = 1}^{n} {f}_{i} {K}_{i} + {( {\sum }_{i = 1}^{n} \frac{{f}_{i}}{{K}_{i}} )}^{- 1} ] , \\ G = \frac{1}{2} [ {\sum }_{i = 1}^{n} {f}_{i} {G}_{i} + {( {\sum }_{i = 1}^{n} \frac{{f}_{i}}{{G}_{i}} )}^{- 1} ] ,$$
wherein Kᵢ is a bulk modulus of the i-th substance in the matrix of the deposit, and Gᵢ is a shear modulus of the i-th substance in the matrix of the deposit;
(3) calculating the compressional wave velocity of a pure natural gas hydrate utilizing a formula
$${V}_{p h} = \sqrt{\frac{E ( 1 - σ )}{ρ ( 1 + σ ) ( 1 - 2 σ )}} ,$$
wherein E is a Young's modulus of the pure natural gas hydrate, ρ is a density of the pure natural gas hydrate, and σ is a Poisson's ratio of the pure natural gas hydrate; wherein the Young's modulus is obtained by a formula
$$E = \frac{9 K G}{3 K + G} ,$$
and the Poisson's ratio is obtained by a formula
$$σ = \frac{3 K - 2 G}{2 ( 3 K + G )} ;$$
(4) multiplying both sides of the equation by
$$\frac{1}{{ρ}_{b} {V}_{b}^{2}} = \frac{Φ ( 1 - {S}_{h} )}{{ρ}_{w} {V}_{p w}^{2}} + \frac{Φ {S}_{h}}{{ρ}_{h} {V}_{p h}^{2}} + \frac{1 - Φ}{{ρ}_{m} {V}_{p m}^{2}}$$
by
$$\frac{1}{{ρ}_{b}}$$
to obtain an equation
$$\frac{1}{{( {ρ}_{b} {V}_{b} )}^{2}} = \frac{Φ ( 1 - {S}_{h} )}{{ρ}_{b} {ρ}_{w} {V}_{p w}^{2}} + \frac{Φ {S}_{h}}{{ρ}_{b} {ρ}_{h} {V}_{p h}^{2}} + \frac{1 - Φ}{{ρ}_{b} {ρ}_{m} {V}_{p m}^{2}} ;$$
wherein a compressional wave impedance of the deposit containing the natural gas hydrate is Z_{b}=ρ_{b}V_{b}, the compressional wave impedance of the fluid is Z_{w}=ρ_{w}V_{pw}, a compressional wave impedance of the pure natural gas hydrate is Zₕ=ρₕVₚₕ, a compressional wave impedance of the matrix of the deposit is Zₘ=ρₘVₚₘ, and then the equation
$$\frac{1}{{( {ρ}_{b} {V}_{b} )}^{2}} = \frac{Φ ( 1 - {S}_{h} )}{{ρ}_{b} {ρ}_{w} {V}_{p w}^{2}} + \frac{Φ {S}_{h}}{{ρ}_{b} {ρ}_{h} {V}_{p h}^{2}} + \frac{1 - Φ}{{ρ}_{b} {ρ}_{m} {V}_{p m}^{2}}$$
can be expressed as an equation
$$\frac{1}{{( {Z}_{b} )}^{2}} = \frac{Φ ( 1 - {S}_{h} )}{( {ρ}_{b} / {ρ}_{w} ) {( {Z}_{w} )}^{2}} + \frac{Φ {S}_{h}}{( {ρ}_{b} / {ρ}_{h} ) {( {Z}_{h} )}^{2}} + \frac{1 - Φ}{( {ρ}_{b} / {ρ}_{m} ) {( {Z}_{m} )}^{2}} ;$$
multiplying both sides of an equation ρ_{b}=(1−Sₕ)Φρ_{w}+ΦSₕρₕ+(1−Φ)ρₘ by
$$\frac{1}{{ρ}_{w}}$$
to obtain an equation ρ_{b}/ρ_{w}=(1−Sₕ)Φ+ΦSₕρₕ/ρ_{w}+(1−Φ)ρₘ/ρₕ, multiplying both sides of an equation ρ_{b}=(1−Sₕ)Φρ_{w}+ΦSₕρₕ+(1−Φ)ρₘ by
$$\frac{1}{{ρ}_{h}}$$
to obtain an equation ρ_{b}/ρₕ=(1−Sₕ)Φρ_{w}/ρₕ+ΦSₕ+(1−Φ)ρₘ/ρₕ, and multiplying both sides of an equation ρ_{b}=(1−Sₕ)Φρ_{w}+ΦSₕρₕ+(1−Φ)ρₘ by
$$\frac{1}{{ρ}_{m}}$$
to obtain an equation ρ_{b}/ρₘ=(1−Sₕ)Φρ_{w}/ρₘ+ΦSₕρₕ/ρₘ+(1−Φ), and setting C_{bw}=ρ_{b}/ρ_{w}, C_{bh}=ρ_{b}/ρₕ, C_{bm}=ρ_{b}/ρₘ, and C_{bh} [symbol] C_{bw} [symbol] 1 [symbol] C_{bm};
(5) substituting C_{bw}, C_{bh} and C_{bm} into an equation
$$\frac{1}{{( {Z}_{b} )}^{2}} = \frac{Φ ( 1 - {S}_{h} )}{( {ρ}_{b} / {ρ}_{w} ) {( {Z}_{w} )}^{2}} + \frac{Φ {S}_{h}}{( {ρ}_{b} / {ρ}_{h} ) {( {Z}_{h} )}^{2}} + \frac{1 - Φ}{( {ρ}_{b} / {ρ}_{m} ) {( {Z}_{m} )}^{2}} ,$$
to obtain a formula
$$\frac{1}{{( {Z}_{b} )}^{2}} = \frac{Φ ( 1 - {S}_{h} )}{{{C}_{b w} ( {Z}_{w} )}^{2}} + \frac{Φ {S}_{h}}{{{C}_{b h} ( {Z}_{h} )}^{2}} + \frac{1 - Φ}{{{C}_{b m} ( {Z}_{m} )}^{2}}$$
for calculating the saturation of the natural gas hydrate by using a Wood wave impedance method, wherein the compressional wave impedance Z_{b} of the deposit containing the natural gas hydrate is obtained by compressional wave impedance inversion, and the compressional wave impedance Z_{w} of the fluid and the compressional wave impedance Zₕ of the pure natural gas hydrate are calculated by measuring relevant elastic parameters in a laboratory; the compressional wave impedance Zₘ of the matrix is calculated on the basis of drilling data and measurement data of the relevant elastic parameters measured in the laboratory, and the porosity Φ is obtained by utilizing a logging interpretation technique.

2. A method for estimating saturation of a natural gas hydrate contained in a deposit, the method comprising:
obtaining a compressional wave impedance Z_{b} of the deposit containing the natural gas hydrate by compressional wave impedance inversion;
calculating a compressional wave impedance Zₕ of the natural gas hydrate in a pure state by laboratory measurement of at least one elastic parameter;
calculating a compressional wave impedance Z_{w} of a fluid by laboratory measurement of at least one elastic parameter;
calculating a compressional wave impedance Zₘ of the deposit on the basis of drilling data and laboratory measurement data of at least one elastic parameter;
obtaining a porosity Φ of the deposit utilizing a logging interpretation technique;
calculating the saturation of the natural gas hydrate in the deposit utilizing a formula
$$\frac{1}{{( {Z}_{b} )}^{2}} = \frac{Φ ( 1 - {S}_{h} )}{{{C}_{b w} ( {Z}_{w} )}^{2}} + \frac{Φ {S}_{h}}{{{C}_{b h} ( {Z}_{h} )}^{2}} + \frac{1 - Φ}{{{C}_{b m} ( {Z}_{m} )}^{2}} ,$$
wherein Sₕ represents a proportion of the natural gas hydrate in a pore space of the deposit, C_{bw}=ρ_{b}/ρ_{w}, C_{bh}=ρ_{b}/ρₕ, and C_{bm}=ρ_{b}/ρₘ, wherein ρ_{b} represents a bulk density of the deposit containing the natural gas hydrate, ρ_{w} represents a density of fluid contained in the deposit, ρₕ represents a density of the natural gas hydrate in a pure form, and ρₘ represents a density of the deposit as a matrix, and wherein C_{bh} [symbol] C_{bw} [symbol] 1 [symbol] C_{bm}; and
outputting the calculated saturation of the natural gas hydrate in the deposit.
